(19) United States
(12) Patent Application Publication
(10) Pub. No.: US 2021/0292696 A1
(43) Pub. Date: Sep. 23, 2021

(54) GRAVITY FLOW CELL CULTURE DEVICES, SYSTEMS, AND METHODS OF USE THEREOF

(71) Applicant: Corning Incorporated, Corning, NY (US)

(72) Inventors: Gregory Roger Martin, Acton, ME (US); Allison Jean Tanner, Portsmouth, NH (US)

(21) Appl. No.: 16/332,608

(22) PCT Filed: Sep. 21, 2017
(86) PCT No.: PCT/US2017/052765

§ 371 Date: Mar. 12, 2019

Related U.S. Application Data
(60) Provisional application No. 62/398,266, filed on Sep. 22, 2016.

(51) Int. Cl. C12M 1/00; C12M 1/24; C12M 1/32; C12M 3/00

(52) U.S. Cl. CPC C12M 23/58; C12M 23/08; C12M 29/10; C12M 23/44; C12M 23/12

(57) ABSTRACT

Multi-layer cell culture devices and systems for culturing suspension and adherent cells via continuous gravity flow of culture media without the use of motorized equipment minimize the risk of contamination of the cell culture. A multi-layer cell culture device includes at least first and second chambers configured to contain fluid and to be fluidly connected such that fluid in the first chamber unidirectionally and continuously flows by gravity flow from the first chamber and into the second chamber. A multi-layer cell culture device can further include a third chamber configured to contain fluid and positioned below the second chamber such that fluid in the second chamber unidirectionally and continuously flows by gravity flow from the second chamber and into the third chamber. Methods of culturing cells using these devices and systems are also described.

### CROSS-REFERENCE TO RELATED APPLICATIONS

This application claims the benefit of priority of U.S. Provisional Application Ser. No. 62/398,266 filed on Sep. 22, 2016 the content of which is relied upon and incorporated herein by reference in its entirety.

### FIELD OF THE INVENTION

The invention relates to continuous gravity flow cell culture devices and systems, as well as methods for culturing cells using the devices and systems.

### BACKGROUND

One method of providing nutrients and diluting waste products of cells in culture is to culture the cells in suspension. The density of cells in suspension can be quite high, but to achieve high densities of cells, mixing, stirring, shaking and/or sparging of gasses may be required. These activities can cause shear which is a force generally detrimental to the cells in culture. Not all cell types can be adapted to suspension culture, so microcarriers may need to be employed. All of the equipment required can occupy a large volumetric footprint that may be limited by the space available, and separating cells from microcarriers is problematic.

Another method of providing nutrients and diluting waste products of cells in culture is to culture the suspension cells in a compartmentalized vessel where the cells are grown over a gas permeable substrate and/or separated from a large compartment of media by a semi-permeable or dialysis membrane. The nutrients diffuse through the dialysis membrane toward the cells while the waste moves in the opposite direction due to the concentration gradients that form on either side of the membrane. Protein products produced by the cells also remain in the compartment with the cells enabling concentration of the recombinant protein. The CELLine Flask (Integra Biosciences) is one such vessel. However, this is not useful for adherent cells.

Perfusion is another method to provide fresh nutrients for cells in culture. This involves mechanical equipment, such as a circulation pump. There is a wide variety of pumps available with syringe pumps and peristaltic pumps powering most perfusion cell culture systems. The Corning® CellCube® and E-Cube™ systems are perfusion systems that use circulation pumps.

In some cases, initial introduction of media, intermittent bolus feeding, and final removal of nutrient medium and cells occurs by gravity through tubing, as is done with the HYPERStack® and CellSTACK© vessels. However, the design of these vessels does not enable continuous feeding over time.

Other methods of feeding cells in microplates occurs through hydrostatic pressure. In such systems, a first well is at greater pressure than subsequent wells, creating flow through a “wicking” material from a feeding well through a culture well to a waste well. This is the method of operation of the Corning® FloWell™ plates.

A system permitting a simple, non-mechanized method for continuous feeding of cells in culture is not commercially available. This type of system becomes more necessary as geometries enabling the culture of many more cells per square centimeter (e.g., as spheroids) become available, or with cell types such as T-cells that utilize greater volumes of nutrients on a per cell basis. To meet the requirements of T-cells, for example, or an increased number of cells per volumetric footprint, the frequency of feedings would need to increase, adding to the workload of the cell culturist. Each intervention also increases the risk of contaminating the cell culture.

### SUMMARY

Described herein are multi-layer cell culture devices and systems for culturing suspension and adherent cells via continuous gravity flow of culture media without the use of motorized equipment that minimize the risk of contamination of the cell culture. Methods of culturing cells using these devices and systems are also described below.

In an aspect, the disclosure provides a multi-layer cell culture device including: a first chamber configured to contain fluid and including at least a first aperture for introducing or removing fluid from the first chamber; and a second chamber positioned below the first chamber. The second chamber is configured to contain fluid and includes at least a first aperture for introducing or removing fluid from the second chamber. The first and second chambers are configured to be fluidly connected such that fluid in the first chamber unidirectionally and continuously flows by gravity flow from the first chamber and into the second chamber.

A multi-layer cell culture device can further include a third chamber configured to contain fluid and positioned below the second chamber. In this device, the third chamber includes at least a first aperture for introducing or removing fluid from the third chamber, and the second and third chambers are configured to be fluidly connected such that fluid in the second chamber unidirectionally and continuously flows by gravity flow from the second chamber and into the third chamber. Typically, the first chamber is a feed chamber, the second chamber is a culture chamber, and the third chamber is a waste chamber. The first chamber can be substantially parallel to the second chamber. The second chamber can be substantially parallel to the third chamber. The first, second, and third chambers can be substantially parallel to each other. In a multi-layer cell culture device, the fluid unidirectionally and continuously flows by gravity flow over a period of, for example, two or more days.

In an embodiment of a multi-layer cell culture device, at least one of the first and second chambers includes a vent. In an embodiment of a multi-layer cell culture device having at least three chambers, at least one of the first, second and third chambers includes a vent.

In an embodiment of a multi-layer cell culture device, the first chamber can further include a cell-retention apparatus for preventing cells in the first chamber from entering the second chamber. Similarly, the second chamber can further include a cell-retention apparatus for preventing cells in the second chamber from entering the third chamber. The first chamber can contain culture media and a first population of cells, and the second chamber can contain culture media and a second population of cells. For example, the first population of cells can include peripheral blood mononuclear cells (PBMCs) and the second population of cells can include T cells. As another example, the first population of cells can include fibroblasts and the second population of cells can include stem cells. The second chamber can include wells for confinement of cells for spheroid cell culture. In an embodiment of a multi-layer cell culture device, the second chamber has at least one interior surface coated with a material that prevents binding of cells to the at least one interior surface. In another embodiment, the second chamber has a substantially planar horizontal surface for supporting growth of attachment-dependent cells.

In a multi-layer cell culture device as described herein, the chambers can be fluidly connected by at least one connector including tubing. In such an embodiment, a first cap can cover the at least first aperture for introducing or removing fluid from the first chamber, the first cap having at least a first coupler for receiving a first end of the tubing, and a second cap can cover the at least first aperture for introducing or removing fluid from the second chamber, the second cap having at least a first coupler for receiving a second end of the tubing. The first cap can include a second coupler for venting gas from the first chamber, and the second cap can include a second coupler for venting gas from the second chamber. A flow regulator can be operably connected to the tubing. A flow regulator can be, for example, a valve. In one example of a multi-layer cell culture device, the chambers are fluidly connected by at least one connector, and at least one flow regulator (e.g., a valve) is operably connected to the at least one connector.

In an embodiment of a multi-layer cell culture device, the first chamber includes a second aperture for introducing or removing fluid from the first chamber, and the second chamber includes a second aperture for introducing or removing fluid from the second chamber. The second apertures can be, for example, clamped, closed, covered or sealed. The second apertures can each be operably connected to a vent. In some embodiments, the first chamber is a bag and the first and second chambers are fluidly connected by a connector including tubing. A flow regulator can be operably connected to the tubing. In an embodiment of a multi-layer cell culture device, the second aperture of the second chamber is operably connected to a vent.

In an embodiment of a multi-layer cell culture device, the first and second chambers can be fluidly connected by a cannula fluidly connected to the first chamber and at least a first septum fluidly connected to the second chamber. In this embodiment, the first and second chambers are positioned such that the at least first septum is penetrated by the cannula. In such an embodiment, the first chamber can include a first end and a second end and an interior inclined surface at one of the two ends relative to an interior surface at the other of the two ends. In this embodiment, the interior inclined surface and the cannula are at opposite ends of the first chamber. The first chamber can include a second aperture for introducing or removing fluid from the first chamber that is covered by a first cap, and the second chamber can include a second aperture for introducing or removing fluid from the second chamber that is covered by a second cap.

In an embodiment of a multi-layer cell culture device, a surface between the first chamber and the second chamber has an aperture disposed therein and the first chamber and the second chamber are fluidly connected by the aperture disposed in the surface between the first and second chambers and a valve positioned partially internal to the first chamber and extending through the aperture disposed in the surface between the first and second chambers. In such an embodiment, the first chamber can include a first end and a second end and an interior inclined surface at one of the two ends relative to an interior surface at the other of the two ends. In this embodiment, the interior inclined surface and the aperture disposed in the surface between the first and second chambers are at opposite ends of the first chamber. The first chamber can include a second aperture for introducing or removing fluid from the first chamber that is covered by a first cap, and the second chamber can include a second aperture for introducing or removing fluid from the second chamber that is covered by a second cap. In such an embodiment, at least one of the first cap and the second cap can include a vent for venting gas from at least one of the first and second chambers.

In an embodiment of a multi-layer cell culture device, the first chamber can include a first end and a second end and an interior inclined surface at one of the two ends relative to an interior surface at the other of the two ends. In this embodiment, the interior inclined surface and the at least first aperture for introducing or removing fluid from the first chamber are at opposite ends of the first chamber.

The first and second chambers can be flasks. In an embodiment of a multi-layer cell culture device having at least three chambers, the first, second, and third chambers can be flasks.

A multi-layer cell culture device can be manufactured or assembled by, for example, injection molding, blow molding, thermoforming, laser welding, ultrasonic welding, adhesive bonding, thermal bonding, etc.

The first and second chambers can be integrated within a single vessel. In an embodiment of a multi-layer cell culture device having at least three chambers, the first, second and third chambers can be integrated within a single vessel.

In another aspect, the disclosure provides a method of culturing cells. The method includes adding at least a first population of cells and tissue culture media to a multi-layer cell culture device as described herein.

In a further aspect, the disclosure provides a method of culturing cells including adding a first population of cells, a second population of cells, and tissue culture media to a multi-layer cell culture device as described herein. In one example, the first population of cells are PBMCs and the second population of cells are T cells. In another example, the first population or the second population of cells are spheroid-forming cells.

Unless otherwise defined, all technical terms used herein have the same meaning as commonly understood by one of ordinary skill in the art to which this invention belongs.

As used herein, the term “vent” refers to an opening in a chamber, vessel, etc., through which gas can pass into or out of the chamber, vessel, etc. While other fluids (e.g., liquids) may be capable of passing through a vent, a vent is primarily intended for the transfer of gasses.

By the terms “connection” and “connector” is meant anything that connects, joins, or links.

As used herein, the term “valve” refers to a device for regulating the passage of fluid (liquid, gas) through a passage, for example, through an aperture, port, vent, or tube.

By the term “open system” is meant a system that is directly open to the atmosphere when one accesses a chamber of a multi-layer cell culture device.

As used herein, the term “closed system” means a system that is not directly open to the atmosphere when one accesses a chamber of a multi-layer cell culture device.

By the term “pseudo-closed system” is meant a system in which a chamber can be accessed by opening either to the atmosphere or not.

The term “spheroid” as used herein refers to a three-dimensional cell cluster including a number of aggregated cells. Spheroids can be generated from different cell types including primary cells, cell lines, and stem cells.

In use, the first chamber contains fluid, e.g., culture media, that provides nutrients to cells contained within the second chamber. Thus, the first chamber is sometimes referred to herein as the “feed chamber” while the second chamber is sometimes referred to herein as the “culture chamber.” In some embodiments, cells are contained within both the first (feed) and second (culture) chambers. In those embodiments in which a device includes a third chamber, this third chamber typically contains waste fluid, and is thus sometimes referred to herein as the “waste chamber.”

Although devices, systems and methods similar or equivalent to those described herein can be used in the practice or testing of the present invention, suitable devices, systems and methods are described below. All publications, patent applications, and patents mentioned herein are incorporated by reference in their entirety. In the case of conflict, the present specification, including definitions, will control. The particular embodiments discussed below are illustrative only and not intended to be limiting.

### BRIEF DESCRIPTION OF THE DRAWINGS

FIG. 1A shows a schematic side view of a multi-layer cell culture device including three chambers, a flow control valve (a flow regulator), and an enlarged schematic of the flow control valve. In this embodiment, the device is a closed system.

FIG. 1B shows a schematic front view of the multi-layer cell culture device of FIG. 1A.

FIG. 2 shows a schematic side view of another embodiment of a multi-layer cell culture device including three chambers, a flow control valve (a flow regulator), and an enlarged schematic of the flow control valve. In this embodiment, the device is a closed system. Feeder cells are cultured in the feed chamber and spheroids are cultured in the culture chamber which has wells for confinement of cells for spheroid cell culture disposed along the bottom of the interior of the culture chamber.

FIG. 3 shows a schematic side view of another embodiment of a multi-layer cell culture device including three chambers, a flow control valve (a flow regulator) and a bag (referred to herein as a “feed bag”) as the feed chamber. In this embodiment, spheroids are shown cultured in the culture chamber which has wells for confinement of cells for spheroid cell culture disposed along the bottom of the interior of the culture chamber. The device is a closed system.

FIG. 4 shows a schematic side view of another embodiment of a multi-layer cell culture device assembled from traditional, commercially available cell culture vessels. The device includes three chambers and is a closed system.

FIG. 5 shows a schematic side view of another embodiment of an multi-layer cell culture device in which flow between chambers occurs through cannula that have punctured a septum in the adjacent chamber. This device is an open system. In this embodiment, spheroids are shown cultured in the culture chamber which has wells for confinement of cells for spheroid cell culture disposed along the bottom of the interior of the culture chamber.

FIG. 6 shows a schematic side view of another embodiment of a multi-layer cell culture device including three chambers and a flow control valve (a flow regulator), and illustrating four mechanisms for venting the chambers: 1) a vent fluidly connected to the feed chamber, 2) a vent fluidly connected to the feed chamber that is plumbed to the other chambers, 3) a vent disposed within the cap of the culture chamber, and 4) a vent fluidly connected to a cap or coupler.

FIG. 7 shows an enlarged schematic side view of the flow control valve (a flow regulator) of FIG. 6. In a typical embodiment, the valve is turned to increase or decrease engagement of the valve in the top of the chamber below, which in turn increases or decreases the flow of medium from one chamber to the next.

FIG. 8A shows a schematic front view of another embodiment of a multi-layer cell culture device featuring internal venting. FIG. 8B shows an alternative embodiment of FIG. 8A.

### DETAILED DESCRIPTION

Described herein are multi-layer cell culture devices through which a fluid (e.g., culture media) continuously flows by gravity flow (“gravity-feed”) which provide for continuous feeding of cells cultured therein. Cell culture systems including these devices, methods of culturing cells using these systems and devices, and methods of making these devices are also described. The gravity-feed culture systems and devices described herein provide a less labor-intensive method to feed cells fresh or conditioned medium, requiring no motorized equipment. Systems and devices as described herein can be used to culture adherent cells or suspended (suspension) cells, and can also be used to co-culture different cell types (e.g., two or more different cell types or cell populations) together (e.g., while allowing for physical separation of cell types or cell populations). They can also be used for media-conditioning by feeder cells. In some embodiments, the devices can be assembled as an integral unit having multiple chambers within (e.g., for feeding, culture, waste, etc.), or as separate vessel modules (e.g., feeding module, culture module, waste module, etc.). Systems and devices described herein provide stacked flow-through operation that permits the addition of chemical entities to cells within a culture device. In some embodiments, cells within the device can be assessed (e.g., assessed kinetically for altered physiology (e.g., due to the addition and/or removal of the chemical entities)). Devices as described herein can be manufactured as open systems, as closed systems, or as pseudo-closed systems.

The gravity-feed systems and devices described herein enable continuous or intermittent feeding without extraneous equipment or invasion of (entry into) the chambers of a device after initial set up of the culture device and/or system. These systems and devices are flexible, adaptable, and modifiable to accommodate a vast array of user requirements. Modular systems that are connected (e.g., by a user) in various arrangements and with various accessories and modifications are within the scope herein. Devices that are manufactured as a single device having two or more integrated chambers are also within the scope herein. Interior surfaces of a device can be made of any suitable materials, including inert materials, low or non-adhesive materials, gas permeable or impermeable materials, transparent or opaque materials, etc. In some embodiments (e.g., for culturing suspension cells, spheroids, etc.), one or more interior surfaces of a device can be treated or coated with a material that discourages or prevents binding of cells to the surface or enhances adhesion of cells to the surface.

In some embodiments, flow from one chamber to the next is controlled solely by the orientation of the respective chambers (e.g., relative height/altitude) and the size (e.g., internal diameter of the apertures and/or connectors and/or couplers). In other embodiments, one or more flow regulators, e.g., valves, are utilized to control the flow rate. In some embodiments, flow from one chamber to the next is controlled by a combination of the orientation (e.g., relative height/altitude) of the chambers and one or more flow regulators (e.g., valves). The scope is not limited by the type of flow regulator (e.g., valve) or means of flow restriction.

The below described preferred embodiments illustrate adaptations of these devices, systems and methods. Nonetheless, from the description of these embodiments, other aspects of the invention can be made and/or practiced based on the description provided below.

### Cell Culture Methods

Methods involving conventional cell culturing techniques are described herein. Such techniques are generally known in the art. Methods of culturing spheroids from cell lines, primary culture or primary isolate samples are known in the art and are described, for example, in U.S. Patent Application Pub. Nos. 2015/0376566, 2014/0322806, and 2009/0325216. Methods of culturing suspension cells such as T-cells are also known in the art and are described, for example, in U.S. Pat. No. 8,034,334 and U.S. Application Pub. Nos. 2016/0010058, 2012/0244133, and 2006/0263881. All of these references are incorporated by reference herein.

### Multi-Layer Cell Culture Devices

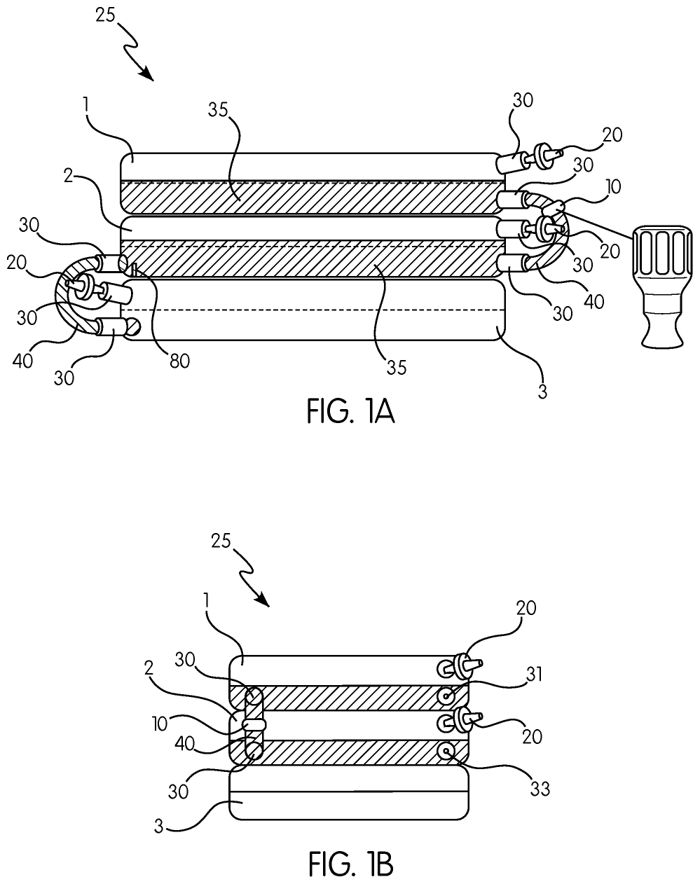

Devices and systems include two or more (e.g., two, three, four, five, six, seven, eight, nine, ten, etc.) chambers arranged vertically (e.g., at different heights) and connected in series (parallel connections may also be utilized in certain embodiments (e.g., a feed chamber that provides fluid to two or more culture chambers)) to provide flow from upstream chambers to downstream chambers. In some embodiments, the relative height of two chambers is designed (e.g., by a manufacturer or a user) or adjusted (e.g., by a user) to provide a desired flow rate or range of flow rates (e.g., optionally, further adjustable by a flow regulator such as a valve). Designing or changing the height of the liquid-containing chamber relative to the cell-containing chamber alone can provide a desired flow rate. In some embodiments, chambers are stacked one atop another (with or without spaces in between). In some embodiments, chambers are supported by a rack, shelving system, or other support structure. Chambers may be aligned in the horizontal x- and/or y-dimensions, while being appropriately offset in the vertical z-dimension, or may also be offset in the horizontal dimensions. FIGS. 1-8 depict several embodiments of the devices and systems described herein.

FIG. 1A and FIG. 1B show a schematic side view of a closed system, three-chamber gravity-feed culture device (a multi-layer cell culture device) including a flow control valve, and an enlarged schematic of the flow control valve, and a schematic front view of the device, respectively. As shown in FIGS. 1A and 1B, one embodiment of a multi-layer cell culture device 25 includes a first chamber 1 configured to contain fluid 35. The first chamber 1 includes at least a first aperture for introducing or removing fluid from the first chamber 1. This device also includes a second chamber 2 positioned below the first chamber 1, the second chamber 2 configured to contain fluid 35 and including at least a first aperture for introducing or removing fluid from the second chamber 2. This device further includes at least a first connector coupled to the at least first apertures of the first and second chambers 1, 2 and fluidly connecting the first and second chambers 1, 2 such that fluid 35 in the first chamber 1 unidirectionally and continuously flows by gravity flow through the at least first connector and into the second chamber 2. The at least first connector, or a flow regulator 10 operably connected to the at least first connector, is positioned to encourage gravity flow and regulates the gravity flow. In this embodiment, the at least first connector includes tubing 40. A coupler 30 on the first chamber 1 that interfaces with the at least first aperture on the first chamber 1 receives a first end of the tubing 40 and a coupler 30 on the second chamber 2 that interfaces with the at least first aperture on the second chamber 2 receives the other end (a second end) of the tubing 40. A coupler is any structure to facilitate the transfer of fluid (e.g., continuous transfer, constant rate transfer, etc.) into and/or out of an aperture or port. Such structures include, for example, a spout, funnel, mouth, stem, nozzle, etc. In some embodiments, the coupler is structured to allow attachment of a component (e.g., conduit for fluid transfer, valve, etc.) to a chamber. In some embodiments, the coupler 30 includes or is connected to a structure 20 (e.g., seal, cap, lid, vent, plug, valve) for preventing fluid transfer through an aperture, such components or structures can be reversible (e.g., can be repeatedly opened and closed) or irreversible (e.g., can be opened or closed only once). Flow into or from an aperture may be regulated only by the size of the aperture and orientation of the chamber and/or environment on either side of the aperture, or may be regulated by one or more types of flow regulators (e.g., flow valves), or a combination thereof. In FIG. 1A, a flow regulator 10 is operably connected to the tubing 40, and the flow regulator 10 is shown as a valve 10 in this embodiment. In this embodiment, flow from one chamber to a chamber below is regulated or controlled by the valve 10 constricting the tubing 40.

In FIG. 1A, the device 25 includes a third chamber 3 configured to contain fluid and positioned below the second chamber 2. The third chamber 3 includes at least a first aperture for introducing or removing fluid from the third chamber 3. The second chamber 2 includes a second aperture for introducing or removing fluid from the second chamber 2, and the second and third chambers are fluidly connected by a second connector that includes tubing 40 such that fluid in the second chamber 2 unidirectionally and continuously flows by gravity flow through the second connector and into the third chamber 3. The second connector is positioned to encourage gravity flow and may regulate the gravity flow. In this embodiment, the second connector which includes tubing 40, is positioned at an opposite end of the second chamber 2 from the end of the second chamber 2 having the at least first connector. A coupler 30 on the second chamber 2 that interfaces with the second aperture on the second chamber 2 receives a first end of the tubing 40 and a coupler 30 on the third chamber 3 that interfaces with the at least first aperture on the third chamber 3 receives the other end (a second end) of the tubing 40. Flow into or from an aperture may be regulated only by the size of the aperture and orientation of the chamber and/or environment on either side of the aperture, or may be regulated by one or more types of flow regulators (e.g., flow valves), or a combination thereof.

Also shown in FIG. 1A is a cell-retention apparatus 80 positioned in the interior of the second chamber 2 to prevent cells in the second chamber 2 from entering the third chamber 3. The cell-retention apparatus 80 is positioned in suitable proximity to the second aperture of the second chamber 2 for retaining cells. One example of a cell-retention apparatus 80 is a weir. A multi-layer cell culture device as described herein may include one or more weirs, for example, in a particular chamber to limit liquid to only a portion of the chamber. In some embodiments, once the volume of the chamber exceeds a particular height, the weir is breached and liquid flows to the other side of the weir. In some embodiments, a weir is used to prevent outflow of liquid, except in the event of overfilling. In other embodiments, a weir prevents suspension cells from exiting a chamber, while allowing the outflow of liquid. In embodiments where a multi-layer cell culture device has only first and second chambers (i.e., no third chamber), the second chamber may include a cell-retention apparatus.

Each chamber includes at least one aperture for introducing or removing fluid, but the chambers can include two or more (e.g., two, three, four, five) apertures for introducing or removing fluid (e.g., inlet and outlet ports). As shown in FIG. 1A, the first chamber 1 has another coupler 30 that interfaces with a second aperture on the first chamber 1, this coupler 30 shown closed or sealed by structure 20. Similarly, the second chamber 2 is shown with another coupler 30 that interfaces with a third aperture on the second chamber 2, this coupler 30 shown closed or sealed by structure 20 (e.g., a closure component). As with the first and second chambers 1, 2, the third chamber 3 is shown with another coupler 30 that interfaces with a second aperture on the third chamber 3. This coupler 30 is shown closed or sealed by structure 20. Couplers and apertures can be closed, clamped, sealed or covered by any suitable structures, including, for example, plugs, vents, clamps, etc.

FIG. 1B is a front view of the device 25 showing additional apertures 31, 33 (for introducing or removing fluid) on the first chamber 1 and second chamber 2. Fluid such as cell culture media can be introduced into the first chamber 1, second chamber 2, and third chamber 3 through any one or more of their apertures. An aperture on any one of the first, second and third chambers can be used for introducing or removing cells, media, and/or samples. In one example, a first chamber (feed chamber) can have an additional aperture for adding culture media to the first chamber (feed chamber). By replenishing the feed chamber with media, rather than the culture chamber, in some embodiments, additional media can be added to the system without disruption of the cells cultured therein. Such an additional aperture on the first chamber can also or alternatively be used for adding feeder cells to the first chamber 1 (feeder layer seeding). In some embodiments, the second chamber 2 (culture chamber) may have additional apertures, couplers and connectors for connecting the second chamber 2 to additional feed and/or waste chambers, and/or to other components or vessels (e.g., a supplemental nutrient supply vessel, an assay vessel, etc.). In such embodiments, particularly in embodiments in which feeder cells are used to condition the media, an additional feed chamber can be included in a multi-layer cell culture device as described herein. For example, in such a device, a primary feed chamber transfers unconditioned media into a secondary feed chamber including feeder cells, and the secondary feed chamber transfers conditioned media to the culture chamber.

In the multi-layer cell culture devices described herein, chambers can be oriented such that apertures for introducing fluid (e.g., inlet ports) are on an upper portion of a chamber (e.g., the top of the chamber). In some embodiments, an inlet port of a particular chamber is configured or positioned to deliver liquid (culture media) beneath the liquid line of that particular chamber (e.g., to reduce disruption of the liquid by dripping or splashing). In some embodiments, chambers are oriented such that an aperture for removing fluid (e.g., outlet port) of a particular chamber is configured or positioned on a lower portion of that particular chamber (e.g., the bottom of the chamber).

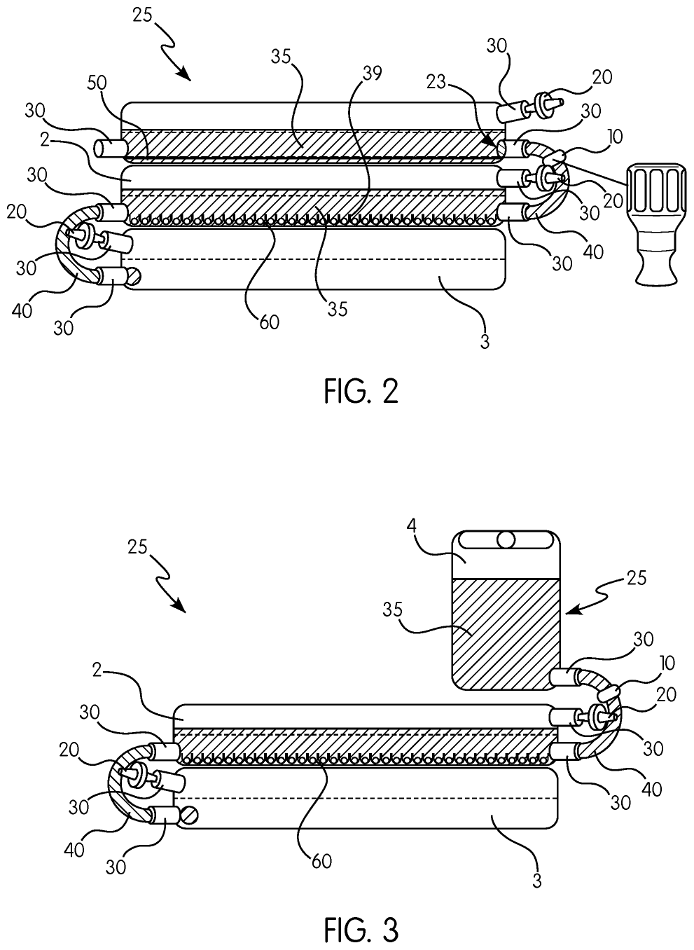

FIG. 2 shows an embodiment of a multi-layer cell culture device 25 having additional features and two populations of cells co-cultured within the first and second chambers 1, 2. A first population of cells 50 (e.g., feeder cells such as fibroblasts) are shown in the first chamber 1 and a second population of cells 60 (typically a second type of cells, e.g., stem cells) are shown in the second chamber 2. Some cell types (e.g., stem cells) perform more realistically if they receive medium that has been conditioned by a second cell type (e.g., fibroblasts). For example, stem cells cultured as spheroids are co-cultured with fibroblasts, as mesenchymal stem cells have been found to have better physiologic characteristics when cultured as spheroids. In such embodiments, a geometry beneficial for spheroid formation is molded, embossed, or attached to the bottom surface of the interior of the second (culture) chamber. FIG. 2 shows wells 39 in the second chamber 2 for confinement of cells 60 for spheroid culture. Another example of co-culturing cells is the use of PBMCs for culturing T cells. In such an embodiment, the first population of cells 50 are PBMCs, and the second population of cells 60 are T cells.

In order to prevent cells 50 in the first chamber 1 (a first population of cells) from entering the second chamber 2, the first chamber 1 can include a cell-retention apparatus. In addition to the use of weirs, as described above, an option to retain suspension cells such as T-cells in the culture chamber is to utilize filter or mesh over the at least first aperture (e.g., outflow port) in the culture chamber. Such a filter prevents the suspension cells from entering the waste chamber (and the waste stream). A filter assembled over the outflow port of the feed chamber is utilized in some embodiments in which feeder cells are cultured in a feed chamber. There is a step in the method for expansion of T-cells with Chimeric Antigen Receptors for which this type of device could be of particular use. The rapid proliferation of transduced T-cells can be induced by culturing the transduced T-cells in the presence of allogeneic irradiated PBMCs, Muromonab-CD3 (OKT3, an immunosuppressant) and Interleukin 2 (IL2, a cytokine signaling molecule). In some embodiments, PBMCs, OKT3 and IL2 are placed in the feed chamber, and the T-cells in the culture chamber. The conditioned medium from the feed chamber flows over time into the T-cell culture chamber to facilitate proliferation, with spent medium flowing into the waste chamber. In the embodiment shown in FIG. 2, the cell-retention apparatus 23 is a mesh material covering the at least first aperture in the first chamber 1 such that cells in the first chamber 1 cannot pass through the at least first aperture and out of the first chamber 1.

In the embodiment shown in FIG. 2, the first chamber 1 contains an additional aperture and an additional coupler 30 at the end of the first chamber 1 opposite to the end of the first chamber 1 having the at least first aperture. In such an embodiment, this additional aperture and coupler can be used for adding feeder cells to the first chamber 1 (i.e., feeder layer seeding).

The chambers of a multi-layer cell culture device (for an integral device, or as separate chambers for a modular device) as described herein can be made of any suitable materials. In some embodiments, chambers are hard-sided (rigid), defined dimension vessels, such as a plate, plate and lid, flask, tube, etc. In embodiments in which the chambers are made of a rigid material, such chambers thus have rigid walls. In other embodiments, one or more of the chambers are soft-sided (pliable), expandable vessels, such as a bag. In typical embodiments, the culture chamber is a hard-sided vessel, while feed and/or waste chambers are either soft- or hard-sided. In some embodiments, a culture chamber is soft-sided such as the embodiment shown in FIG. 3. In the embodiment shown in FIG. 3, the first chamber 4 is a bag that is made of a pliable material (the pliable material can be impermeable to fluids, but permeable to gases, for example). In this embodiment, the at least first connector coupled to the at least first apertures of the first chamber 4 and second chamber 2 and fluidly connecting the first and second chambers 4, 2 such that fluid 35 in the first chamber 4 unidirectionally and continuously flows by gravity flow through the at least first connector and into the second chamber 2, includes tubing 40. A coupler 30 on the first chamber 4 that interfaces with the at least first aperture on the first chamber 4 receives a first end of the tubing 40 and a coupler 30 on the second chamber 2 that interfaces with the at least first aperture on the second chamber 2 receives the other end (a second end) of the tubing 40. In this embodiment, flow (e.g., of culture media) can be controlled by the height of the bag (first chamber 4) above the second chamber 2, and/or with a flow regulator 10 such as a flow control valve. In the embodiment shown in FIG. 3, the device 25 includes a third chamber 3 (waste chamber). In some embodiments, the third chamber (waste chamber) can also be a bag. A bag can be made of any suitable pliable or flexible material. In one embodiment of a multi-layer cell culture device for expansion of suspension cells into multiple chambers, a feed bag (a first chamber) can be positioned above and fluidly connected to a second chamber that contains suspension cells, the second chamber positioned above and fluidly connected to at least a third (e.g., a third, fourth, fifth, sixth, etc.) chamber. As the suspension cells proliferate in the second chamber, a flow regulator valve operably connected to the second and third chambers can be manipulated to allow flow of suspension cells into the third chamber. In this embodiment, the flow and expansion of suspension cells can continue through additional chambers in series.

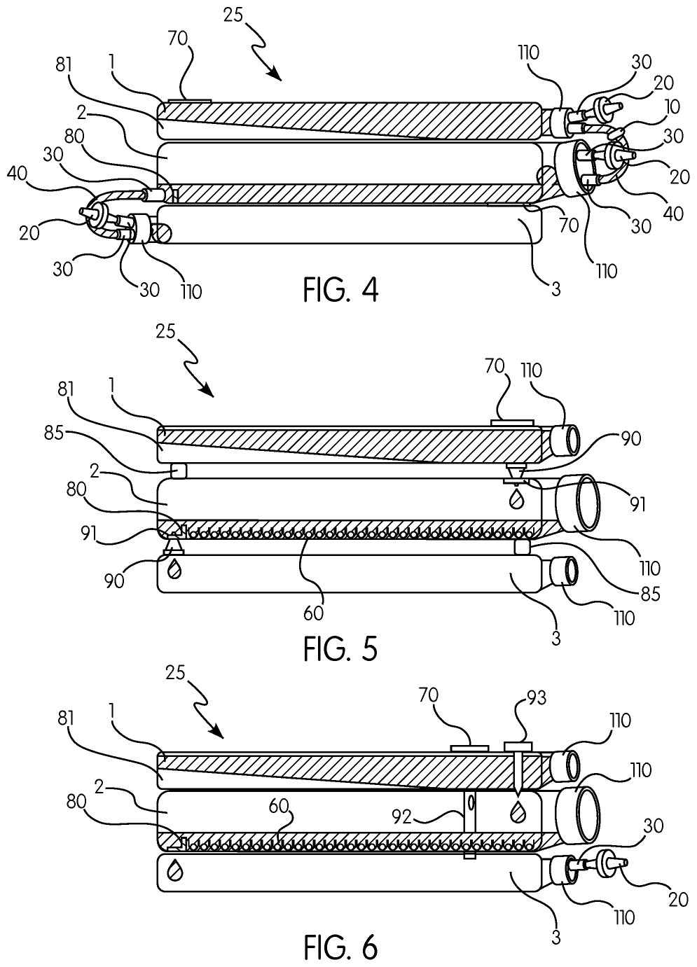

FIG. 4 shows an embodiment of a pseudo-closed system and multi-layer cell culture device 25 involving traditional cell culture vessels and tubing that is attached to the vessels via caps and couplers. In this embodiment, the at least first connector includes tubing 40, a first cap 110 covering the at least first aperture for introducing or removing fluid from the first chamber 1 and having at least a first coupler 30 for receiving a first end of the tubing 40, and a second cap 110 covering the at least first aperture for introducing or removing fluid from the second chamber 2 and having at least a first coupler 30 for receiving a second end of the tubing 40. In this embodiment, the second connector coupled to the second and third chambers 2, 3 includes tubing 40, coupler 30 at the end of the second chamber 2 that is opposite to the end having the at least first aperture of the second chamber 2 and that receives one end of tubing 40, and on the third chamber 3, a third cap 110 covering the at least first aperture of the third chamber 3 and having at least a first coupler for receiving the other end of tubing 40. In this embodiment, the device 25 includes two vent filters 70, one positioned on the top surface or wall of the first chamber 1, and another positioned between the second and third chambers 2, 3. The vent filter 70 connected to the first chamber 1 is fluidly connected to the interior of the first chamber 1 and allows gas to pass out of the first chamber (and out of the device 25). The vent filter 70 positioned between the second and third chambers 2, 3 may be fluidly connected to the interiors of both the second and third chambers 2, 3, such that gas flows between the second and third chambers 2, 3. In other embodiments, instead of vent filters 70 as shown in FIG. 4, caps 110 may include a vent filter or other venting material or apparatus, or one or more couplers 30 may be attached to vents (for example, structures 20 may be vents). Also shown in FIG. 4 is cell-retention apparatus 80 in the second chamber 2 positioned in suitable proximity to the second aperture of the second chamber 2 for retaining cells in the second chamber 2. The cell-retention apparatus 80 is shown as a weir, but a mesh material, for example, could be used instead. Another feature of this embodiment is the first chamber 1 which includes a first end and a second end and an interior inclined surface 81 at one of the two ends relative to an interior surface at the other of the two ends. The interior inclined surface 81 and the at least first aperture for introducing or removing fluid from the first chamber are at opposite ends of the first chamber. This angled (inclined) interior of the first chamber 1 encourages liquid (culture media) flow (gravity flow) through the at least first aperture for introducing or removing fluid from the first chamber 1 and into the at least first connector.

Referring to FIG. 5, the embodiment of a multi-layer cell culture device 25 shown is an open system in which there are spaces between the first, second and third chambers 1, 2, 3. These spaces are created by structures 85, one of which is disposed between the first and second chambers 1, 2, and another of which is disposed between the second and third chambers 2, 3. These structures 85 can be made of any suitable material, and can be formed as part of the chambers or attached thereto. The spaces between the chambers allow gas exchange to gas permeable lower surfaces in the vessels. In this embodiment, the at least first connector includes a cannula 90 fluidly connected to the first chamber 1 and at least a first septum 91 fluidly connected to the second chamber 2, the first and second chambers 1, 2 positioned such that the at least first septum 91 is penetrated by the cannula 90. In this device 25, cannula 90 is disposed in the lower surface or wall of the first chamber 1 and penetrates or pierces the septum 91 that is disposed in or on the upper surface or wall of the second chamber 2. Liquid (e.g., culture media) flows by gravity flow from the first chamber 1 through the cannula 90 and septum 91 and into the second chamber 2. Third chamber 3 has a cannula 90 disposed on its upper surface or wall that penetrates or pierces a septum 91 disposed on the lower surface or wall of the second chamber 2. In an alternative embodiment, this arrangement may be reversed, i.e., the third chamber 3 may have a septum disposed on its upper surface or wall that is penetrated or pierced by a cannula disposed on the bottom surface or wall of the second chamber 2. In either arrangement, liquid flows by gravity flow from the second chamber 2 into the third chamber 3. As shown in this figure, the at least first apertures of the first, second and third chambers 1, 2, 3 are covered by caps 110.

In FIG. 5, vent 70 is shown positioned on or connected to the upper surface or wall of the first chamber 1 for removing gas from the first chamber (and from the device or system, i.e., all of the chambers). As with other embodiments described herein, instead of having vent 70 positioned on or connected to the upper surface or wall of the first chamber 1, a vent filter may be disposed within cap 110 covering the at least first aperture of the first chamber 1. The first chamber 1 is also shown having an interior inclined surface 81 as shown in FIG. 4. The second chamber 2 is shown having cells 60 (e.g., spheroids) that are contained within wells (e.g., spheroid-forming geometry), and a cell-retention apparatus 80 for preventing the cells 60 from entering the third chamber 3.

In some embodiments, chambers include one or more vents to allow intake and/or outlet of gases (e.g., air) from the chambers and/or the device or system. In some embodiments, as gravity draws liquid from a feed chamber into a culture chamber, air in the feed chamber is replaced to prevent negative pressure in the feed chamber reducing the rate of liquid transfer (in the case of a soft-sided feed chamber (e.g., a bag), a vent is not typically necessary for this purpose). A vent in the feed chamber allows air or other gas (e.g., inert gas (e.g., nitrogen or argon), etc.) to be drawn into the chamber to replace the volume of the liquid as it is transferred out. Similarly, as gravity draws liquid from a culture chamber into a waste chamber, the volume in the waste chamber must escape to prevent positive pressure in the waste chamber reducing the rate of liquid transfer (in the case of a soft-sided waste chamber (e.g., a bag), a vent is not typically necessary for this purpose). A vent in the waste chamber allows air or other gas (e.g., inert gas (e.g., nitrogen or argon), etc.) to exit the chamber to provide space in the chamber as liquid is transferred in. A vent is typically an opening (e.g., valve, open aperture, filter-covered aperture, gas-permeable-membrane-covered aperture, etc.) that allows air or other gases to pass. Open and closed systems may have vents. In some embodiments, the vents are covered by a material with 0.2 micron pores to allow gas passage but not bacterial passage.

Vents may include structures (e.g., nozzle, valve, etc.) to facilitate gas transfer and/or to allow attachment of components (e.g., valve, conduit, etc.) to the vent.

In some embodiments, vents include filters, screens, or physical barriers to prevent the introduction of contaminants into the device or system.

Various mechanisms for venting the system are available. Such mechanisms include, for example: 1) an aperture (e.g., on the top or an upper portion of a chamber), which in some embodiments may be covered with a vent filter (e.g., similar to the ROBOFLASK® by Corning); (2) a conduit that runs between volumes (e.g., between chambers, from the external environment to a chamber, combinations thereof, etc.) and has openings in each volume; (3) a filter-covered opening in the cap of a chamber, the opening off-set to one side of the cap and the cap oriented so that the filter material is positioned toward the top to avoid wetting; (4) a vent filter attached to a tubing coupler on each chamber; etc. These vent examples are not limiting. FIG. 6 illustrates several mechanisms for venting (removing gasses from) the chambers and from the device 25. A first mechanism for venting is vent 70 (e.g., an aperture on the top or an upper portion of a chamber covered with a vent filter) shown positioned on or connected to the upper surface or wall of the first chamber 1. Another venting mechanism shown operably connected to the first, second and third chambers 1, 2, 3 is a conduit 92 that runs between (traverses) the chambers and has openings in each chamber (e.g., a plumbed vent tube). A third mechanism is a filter material disposed within one or more of the caps 110 (e.g., a filter-covered opening in the cap of a chamber, the opening off-set to one side of the cap and the cap oriented so that the filter material is positioned toward the top to avoid wetting). A fourth mechanism is structure 20 (a vent or vent filter) attached to coupler 30 on the third chamber 3. The first and second chambers 1, 2 can additionally or alternatively have such coupler/vent arrangements.

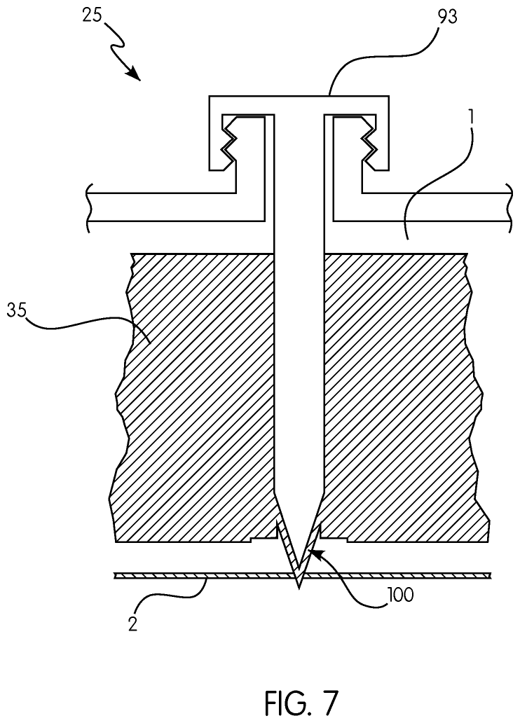

In the device 25 of FIG. 6, the at least first connector includes a flow control valve 93. In this embodiment, the first chamber 1 includes a rigid bottom surface or wall having an aperture disposed therein and the second chamber includes a rigid top surface or wall having an aperture disposed therein such that the bottom surface or wall aperture and the top surface or wall aperture are fluidly and substantially vertically aligned, and the at least first connector includes a valve 93 positioned partially internal to the first chamber 1 and extending through the bottom surface or wall aperture of the first chamber 1 and through the top surface or wall aperture of the second chamber 2. Liquid (e.g., culture media) in the first chamber 1 seeps or passes around the tip or lower end 100 of the valve 93 and into the second chamber 2. In this embodiment, the valve 93 can be turned (rotated) to increase or decrease engagement of the valve 93 in the top surface or wall aperture of the second chamber 2, which in turn increases or decreases the flow (rate of flow) of the liquid from the first chamber 1 into the second chamber 2. In other words, the rate of liquid flow from the first chamber 1 into the second chamber 2 can be regulated or controlled by turning or rotating of the valve 93 such that seepage of the liquid (e.g., culture medium) around the tip 100 of the valve 93 where it meets the upper wall of the second chamber 2 is controlled. The device 25 of FIG. 6 also shows a first chamber 1 having an interior inclined surface 81. The second chamber 2 is shown having cells 60 (e.g., spheroids) that are contained within wells (e.g., spheroid-forming geometry), and a cell-retention apparatus 80 for preventing the cells 60 from entering the third chamber 3. As shown in this figure, the at least first apertures of the first, second and third chambers 1, 2, 3 are covered by caps 110. FIG. 7 is an enlarged schematic side view of the flow control valve 93 of FIG. 6. Valve 93 is shown positioned partially internal to the first chamber 1 and extending through the bottom surface or wall aperture of the first chamber 1 and through the top surface or wall aperture of the second chamber 2. Liquid 35 (e.g., culture media) in the first chamber 1 is shown seeping or passing around the tip or lower end 100 of the valve 93 and into the second chamber 2.

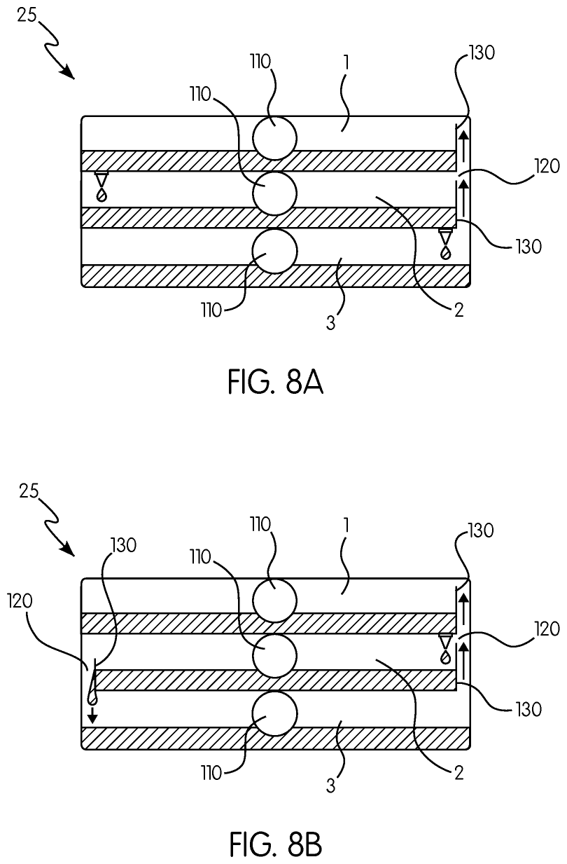

In the embodiments of FIGS. 8A and 8B, an open system multi-layer cell culture device in which the first, second and third chambers 1, 2, 3 are integrated within a single vessel (device) and have internal venting (venting within the vessel or device) is shown. The device 25 has a first chamber 1 including at least a first aperture for introducing or removing fluid from the first chamber 1, a second chamber 2 including at least a first aperture for introducing or removing fluid from the second chamber 2, and a third chamber 3 including at least a first aperture for introducing or removing fluid from the third chamber 3. Each of these apertures is covered by a cap 110. In the device 25 of both FIG. 8A and FIG. 8B, the first and second chambers 1, 2 each have an interior vertical wall 130 positioned such that a continuous space 120 exists between the interior vertical walls 130 and the exterior surface or wall of the device 25 and vertically spans from the first chamber 1 to the third chamber 3. In FIGS. 8A and 8B arrows are shown in the continuous space 120 where internal venting occurs. In FIG. 8A, first chamber 1 and second chamber 2 are shown with a cannula and septum for fluidly connecting the first, second and third chambers 1, 2, 3. In FIG. 8B, a cannula and septum fluidly connect the first and second chambers 1, 2. In this embodiment, the second chamber 2 has a second interior vertical wall 130 creating space 120 that vertically spans the second and third chambers 2, 3, and that acts as a weir to allow liquid to flow from the second chamber 2 to the third chamber 3 but that prevents cells in the second chamber 2 from flowing into the third chamber 3. In an alternative embodiment in which the system is a closed system, the at least first apertures of the first, second and third chambers 1, 2, 3, can be coupled to tubing (as in the embodiments shown in FIGS. 1A, 1B, 2, 3, 4).

The chambers and other structures described herein are formed of any suitable material. Preferably, materials intended to contact cells or culture media are compatible with the cells and the media. Typically, components, including the chambers themselves, are formed from polymeric material(s), such as thermoplastic or thermoset polymers, or glass. Examples of suitable polymeric materials include polystyrene, polymethylmethacrylate, polyvinyl chloride, polycarbonate, polysulfone, polystyrene copolymers, fluoropolymers, polyesters, polyamides, polystyrene butadiene copolymers, fully hydrogenated styrenic polymers, polycarbonate PDMS copolymers, and polyolefins such as polyethylene, polypropylene, polymethyl pentene, polypropylene copolymers and cyclic olefin copolymers, and the like.

In some embodiments, the inner surface of one or more of the chambers, particularly the culture chambers, is low-adherent or non-adherent to cells. The chamber (or inner surface thereof) may be formed from low-adherent or non-adherent (non-adhesive) material or may be coated with low-adherent or non-adherent material. Examples of low-adherent or non-adherent materials include perfluorinated polymers, olefins, or like polymers or mixtures thereof. Other examples include agarose, non-ionic hydrogels such as polyacrylamides, polyethers such as polyethylene oxide, and polyols such as polyvinyl alcohol, or like materials or mixtures thereof. In some embodiments, only chambers or portions of chambers intended for cell contact are low-adherent or non-adherent surfaces. In other embodiments, all chamber surfaces are low-adherent or non-adherent.

Chambers may be of any suitable dimensions. In some embodiments, the dimensions of a chamber are selected based upon the particular use. A larger volume feed and/or waste chamber may be used for longer culture times or cultures require more exchange of fluids. A larger volume culture chamber may be used for larger cell numbers or for cultures requiring a larger media volume. Example widths, lengths, heights, diameters, etc. for chambers include 1 cm, 2 cm, 3 cm, 4 cm, 5 cm, 6 cm, 7 cm, 8 cm, 9 cm, 10 cm, 11 cm, 12 cm, 13 cm, 14 cm, 15 cm, 16 cm, 17 cm, 18 cm, 19 cm, 20 cm, 21 cm, 22 cm, 23 cm, 24 cm, 25 cm, 26 cm, 27 cm, 28 cm, 29 cm, 30 cm, 31 cm, 32 cm, 33 cm, 34 cm, 35 cm, 36 cm, 37 cm, 38 cm, 39 cm, 40 cm, 41 cm, 42 cm, 43 cm, 44 cm, 45 cm, 46 cm, 47 cm, 48 cm, 49 cm, 50 cm, 51 cm, 52 cm, 53 cm, 54 cm, 55 cm, 56 cm, 57 cm, 58 cm, 59 cm, 60 cm, and any suitable ranges there between (e.g., depth of 1-10 cm 1-6 cm, or 1-4 cm; length of 6-40 cm, 8-30 cm, or 10-20 cm; and width of 6-40 cm, 8-30 cm, or 10-20 cm).

Chambers may be of any suitable shape. In some embodiments, the body of the chamber is roughly a rectangular box (e.g., allowing for rounded corners). In some embodiments, variations from such a shape occur at the positions of vents, couplers, connectors, apertures, ports or other features.

In some embodiments, a culture chamber includes wells (e.g., arranged in an array) for the culturing of cells as described above. Wells may be of any suitable size, shape, and/or orientation. In some embodiments, wells are configured for 3D cell culture. In some embodiments, in order to create confinement for cell aggregation, a microwell geometry is used that is very similar (e.g., within 20%, 15%, 10%, 5%, 2%, 1%, or suitable ranges therein) to the size of the maximum desired cell aggregate in diameter, but at least 1 to 2 times the diameter in depth (e.g., 1.0, 1.1, 1.2, 1.3, 1.4, 1.5, 1.6, 1.7, 1.8, 1.9, 2.0, 2.1, 2.2, 2.3, 2.4, 2.5 2.6, 2.6, 2.8, 3.0, 3.5, 4.0, or suitable ranges therein).

There are many different well geometries that are useful for the 3D culture of cells, for example, as aggregates. In some embodiments, cell aggregates are clusters of cells, embryoid bodies, or spheroids. A common geometry to form cell aggregates are hemispheres found on rounded well-bottom microplates. Methods of culturing spheroids from cell lines, primary culture or primary isolate samples are known in the art and are described, for example, in U.S. Patent Application Pub. Nos. 2015/0376566, 2014/0322806, and 2009/0325216. In some embodiments, a non-adhesive (non-adherent) surface is used to prevent the cells from attaching to the surface. A non-adhesive material may be applied after a well or chamber (e.g., in a microplate) is manufactured, or the well or chamber material may have inherent non-attachment characteristics.

Any suitable dimensions for the wells can be used. Well dimensions for use in aggregate cell culture techniques may be on the order of millimeters (e.g., 1 mm to 50 mm). “Microwells” generally have dimensions on the order of micrometers (e.g., <1 mm), and are also used to grow cells as aggregates. Wells may resemble “Aggrewells™” (sold by Stem Cell technologies), which offers a geometry that is an inverse pyramidal shape 400 or 800 micrometers in diameter arrayed in the bottom of standard format microplate wells. Another geometry for growing cells as aggregates is the “Elplasia” microplate with “microspace cell culture” (Kuraray); these plates have square microwells 200 micrometers in diameter arrayed at the bottom of standard format microplate wells that allow cells to aggregate. Various parameters, dimensions, and methods of making microwells for culturing cells as aggregates are understood in the field (e.g., as described in U.S. Pub. No. 2004/0125266; U.S. Pub. No. 2012/0064627; U.S. Pub. No. 2014/0227784; WO2008/106771; herein incorporated by reference in their entireties). U.S. Pat. No. 6,348,999 describes micro relief elements, and how they are constructed, without stating the purpose of these constructs other than as a polymer lens array. U.S. Pat. Nos. 5,151,366, 5,272,084, and 6,306,646 describe vessels with various types of micro relief patterns to increase the surface area for cell attachment on a substrate, and the method of making the culture patterns.

Some commercially available well geometries are conducive to the formation of cell aggregates, but not necessarily conducive to “confinement”. When aggregated cells are not confined, they will usually grow as large as their surroundings will allow. Cell aggregates greater than 150 to 400 micrometers in diameter (depending on the cell type) may form necrotic cores. Necrosis occurs, for example, because the cell mass is inhibitory to the diffusion of nutrients into the center of the aggregate and the metabolic waste out of the center of the aggregate. In some embodiments, in order to create confinement, a microwell geometry is used that is very similar (e.g., within 20%, 15%, 10%, 5%, 2%, 1%, or suitable ranges therein) to the size of the maximum desired cell aggregate in diameter, but at least 1 to 2 times the diameter in depth (e.g., 1.0, 1.1, 1.2, 1.3, 1.4, 1.5, 1.6, 1.7, 1.8, 1.9, 2.0, 2.1, 2.2, 2.3, 2.4, 2.5 2.6, 2.6, 2.8, 3.0, 3.5, 4.0, or suitable ranges therein). In some embodiments, confinement well geometry also allows for exchange of liquid culture medium through perfusion or manual pipetting without lifting the cells out of the confinement wells.

As described above, in some embodiments, a chamber includes more than one aperture for introducing fluid (e.g., inlet port) and more than one aperture for removing fluid (e.g., outlet port). For example, a single (e.g., large) feed chamber including multiple outlet ports is used to provide media to multiple (e.g., smaller) culture chambers. Alternatively, a single culture chamber has inlet ports linked to multiple feed chambers including different media components. In such an embodiment, the flow rates from the various feed chambers is adjusted over time to vary the media composition the cells in the culture chamber receive. Any attachment combination of chambers in series, parallel, or a combination thereof is within the scope herein.

In some embodiments, fluids pass between chambers through a space in a wall shared by both chambers. In some embodiments fluid passes between chambers through directly-mated apertures or ports (e.g., an aperture or port on one chamber is directly aligned with the aperture or port of another chamber). In some embodiments, apertures or ports include structures for facilitating direct mating of apertures or ports (e.g., male and female attachment structures). In some embodiments, a connector or coupler is utilized to secure the mating of two ports. For example, a connector or coupler can attach at a first end to a first port (e.g., an outlet port) and at a second end to a second port (e.g., an inlet port) and deliver fluid (e.g., liquid from a first chamber to a second chamber). A connector or coupler may connect or attach to an aperture or port by any suitable mechanism. In some embodiments, a connector or coupler is removable (e.g., particularly in integrated or modular systems). In some embodiments, a connector or coupler permanently connects two apertures or ports. In some embodiments, a connector or coupler attaches to an aperture or port, and in other embodiments, a connector or coupler runs through or traverses an aperture or port.

In some embodiments, flow of liquid through an aperture or port and/or between chambers is regulated by one or more valves. In some embodiments, a user may select an appropriate flow rate by adjusting one or more valves. In some embodiments, a valve is located in/on or attached to an aperture or port. In other embodiments, a valve is located in/on or attached to a connector or coupler. Any suitable type of valve may find use in embodiments herein. For example, non-limiting types of valves include: a ball valve, disc valve, check valve, choke valve, diaphragm valve, globe valve, needle valve, etc.

In a closed or open system, a single valve may be used to regulate the flow of the entire system (although the use of more than one valve in a closed system is within the scope herein). In some embodiments, each point of transfer between chambers is regulated by a valve.

In some embodiments, a chamber is simply an enclosed volume without any particular structural features other than apertures, ports, vents, etc. In other embodiments, a chamber includes structural features specific to the purpose of the chamber (e.g., feed, culture, waste, etc.) and/or the desired use of the system.

Methods of culturing cells using the multi-layer cell culture devices are described herein. A typical method of culturing cells includes adding at least a first population of cells and tissue culture media to any of the multi-layer cell culture devices described herein. In some methods (e.g., co-culturing methods), a second population of cells is additionally added to the device. For example, a first population of PBMCs and a second population of T cells can be added to the device and cultured therein. As another example, a first population of fibroblasts and a second population of spheroid-forming cells (e.g., stem cells) can be added to the device and cultured therein. In these embodiments, typically the first population of cells and media are added to the first chamber, and the second population of cells and media are added to the second chamber.

### Methods of Making Multi-Layer Cell Culture Devices

A multi-layer cell culture device as described herein can be manufactured by any suitable methods, including injection molding, blow molding, thermoforming, etc. A device as described herein can be assembled using any suitable methods known in the art. Such methods include laser welding, ultrasonic welding, thermal bonding, etc. Materials of construction can be any materials suitable for cell culture vessels, including, for example, thermoplastic polymers, thermoset polymers, glass, etc.

### Other Embodiments

Any improvement may be made in part or all of the devices, systems and method steps. All references, including publications, patent applications, and patents, cited herein are hereby incorporated by reference. The use of any and all examples, or exemplary language (e.g., “such as”) provided herein, is intended to illuminate the devices, systems and methods and does not pose a limitation on the scope of the invention unless otherwise claimed. Any statement herein as to the nature or benefits of the devices, systems and methods or of preferred embodiments is not intended to be limiting, and the appended claims should not be deemed to be limited by such statements. More generally, no language in the specification should be construed as indicating any non-claimed element as being essential to the practice of the devices, systems and methods. The devices, systems and methods include all modifications and equivalents of the subject matter recited in the claims appended hereto as permitted by applicable law. For example, although the devices of FIGS. 1-8 include a third chamber, a third chamber is optional, and a device as described herein may not include a third chamber. A user, for example, may add a third chamber to a device described herein having first and second chambers. In some embodiments the chambers are made of rigid materials, while in other embodiments they are made of pliable materials, and in other embodiments, a combination of rigid and pliable materials. The chambers can be partially or entirely impermeable. Moreover, any combination of the above-described elements in all possible variations thereof is encompassed by the devices, systems and methods unless otherwise indicated herein or otherwise clearly contraindicated by context.

1-48. (canceled)

49. A multi-layer cell culture device comprising:
a first chamber configured to contain fluid and comprising a first end and a second end;
wherein the first chamber comprises a first aperture for introducing or removing fluid from the first chamber;
wherein the first chamber comprises an interior inclined surface;
wherein the interior inclined surface and the first aperture for introducing or removing fluid from the first chamber are at opposite ends of the first chamber;
a second chamber positioned below the first chamber, the second chamber configured to contain fluid, comprising at least a second aperture for introducing or removing fluid from the second chamber;
the first and second chambers fluidly connected such that fluid in the first chamber unidirectionally and continuously flows by gravity flow from the first chamber into the second chamber through the second aperture of the second chamber.

50. The multi-layer cell culture device of claim 49, wherein the second chamber comprises a cell culture surface having an array of microwells for confinement of cells for spheroid cell culture.

51. The multi-layer cell culture device of claim 50 wherein the microwells have rounded well-bottoms.

52. The multi-layer cell culture device of claim 50 wherein the microwells comprise a non-adherent material.

53. The multi-layer cell culture device of any claim 50 wherein the cell culture surface comprises gas permeable material.

54. The multi-layer cell culture device of claim 49 further comprising a third chamber configured to contain fluid and positioned below the second chamber, the third chamber comprising at least a third aperture for introducing or removing fluid from the third chamber, the second and third chambers configured to be fluidly connected such that fluid in the second chamber unidirectionally and continuously flows by gravity flow from the second chamber and into the third chamber.

55. The multi-layer cell culture device of claim 54, wherein the second chamber further comprises a cell-retention apparatus for preventing cells in the second chamber from entering the third chamber.

56. The multi-layer cell culture device of claim 55 wherein the cell-retention apparatus is a weir.

57. The multi-layer cell culture device of claim 53 further comprising structures disposed between the third chamber and the second chamber, to create a gas space below the gas permeable cell culture surface of the second chamber.

58. The multi-layer cell culture device of claim 49 wherein fluid in the first chamber unidirectionally and continuously flows by gravity flow from the first chamber into the second chamber through the second aperture of the second chamber by at least one connector comprising tubing.

59. The multi-layer cell culture device of claim 58 wherein the tubing comprises at least one flow regulator.

60. The multi-layer cell culture device of claim 59, wherein the at least one flow regulator is a valve.

61. The multi-layer cell culture device of claim 49, wherein the first and second chambers are fluidly connected by a cannula fluidly connected to the first chamber and at least a first septum fluidly connected to the second chamber, the first and second chambers positioned such that the at least first septum is penetrated by the cannula.

62. The multi-layer cell culture device of claim 49, wherein the first chamber comprises a first end and a second end and an interior inclined surface at one of the two ends relative to an interior surface at the other of the two ends, wherein the interior inclined surface and the cannula are at opposite ends of the first chamber.

63. The multi-layer cell culture device of claim 49, wherein the first chamber contains culture media and a first population of cells, and the second chamber contains culture media and a second population of cells.

64. The multi-layer cell culture device of claim 62, wherein the first population of cells comprises peripheral blood mononuclear cells (PBMCs) and the second population of cells comprises T cells.

65. The multi-layer cell culture device of claim 63, wherein the first population of cells comprises fibroblasts and the second population of cells comprises stem cells.

66. The multi-layer cell culture device of claim 50, wherein the second chamber has a substantially planar horizontal surface for supporting growth of attachment-dependent cells.

67. The multi-layer cell culture device of claim 55 wherein the first chamber is a feed chamber, the second chamber is a culture chamber, and the third chamber is a waste chamber.

68. The multi-layer cell culture device of claim 55, wherein at least one of the first, second and third chambers comprises a vent.
